(19) United States
(12) Patent Application Publication
(10) Pub. No.: US 2005/0288527 A1
(43) Pub. Date: Dec. 29, 2005

(54) Method for preparing free-flowing crystalline material

(76) Inventor: William Lee Cook, Kingsport, TN (US)

Correspondence Address: Mark L. Davis, P.O. Box 9293, Gray, TN, 37615-9293, US

(21) Appl. No.: 11/156,398

(22) Filed: Jun. 20, 2005

Related U.S. Application Data
(60) Provisional application No. 60/583,344, filed on Jun. 28, 2004.

(51) Int. Cl. C07C 51/42

(52) U.S. Cl. 562/486

(57) ABSTRACT

An improved process for making a high purity, crystalline aromatic dicarboxylic acid product which includes at least one step for crystallizing the product and wherein the crystallized product is thereafter subjected to a drying step. The improved process further includes the steps of storing the crystallized product for a period of time sufficient for at least a portion of residual solvent carried with the dried crystallized product to reside on a surface of the crystalline product; and contacting the stored crystallized product with an inert fluid for a time sufficient to remove at least a portion of the residual solvent in the stored crystallized product. Purified crystalline aromatic dicarboxylic acids suitable for the present improved process include benzene dicarboxylic acid, naphthalene dicarboxylic acid, bibenzoic acid, terephthalic acid, and isophthalic acid. Particularly preferred aromatic dicarboxylic acids include terephthalic acid, and isophthalic acid.

### CROSS REFERENCE TO RELATED APPLICATIONS

Benefit is claimed to the earlier filed application having U.S. Ser. No. 60/583,344 filed Jun. 28, 2004, the entire disclosure of which is incorporated herein by reference.

### BACKGROUND OF THE INVENTION

1. Field of the Invention

The present invention relates to a method for preparing crystalline materials that are free-flowing. More particularly, the present invention relates to a method for preparing high purity, crystalline aromatic dicarboxylic acids that are free-flowing subsequent to purification. More specifically, the present invention relates to a method for preparing high purity, crystalline aromatic dicarboxylic acids that are free-flowing from bulk containers.

2. Background of the Invention

Crystalline aromatic dicarboxylic acids such as: benzene dicarboxylic acid, naphthalene dicarboxylic acid, bibenzoic acid, terephthalic acid and isophthalic acid are used in a variety of processes for the manufacture of various polymers, resins and thermoplastics. Such polymers, resins and thermoplastics are widely accepted for commercial applications. For example, naphthalene dicarboxylic acid, terephthalic acid and isophthalic acid are used extensively in the manufacture of polyesters and polyamides. Polyesters are used to make films, sheets and molded articles which have commercial applications in such fields as automobile parts and food and beverage containers.

Depending upon the desired service, specific aromatic dicarboxylic acid(s) and amounts are used in making polyesters to achieve better tensile strength, lower haze and improved color.

It is well known in the polymer art that crystalline aromatic dicarboxylic acids are generally synthesized by the catalytic oxidation of the corresponding aromatic dialkyl compound. For example, terephthalic acid (TPA) and isophthalic acid (IPA) are produced by the liquid phase oxidation of p-xylene and m-xylene, respectively.

In making polyesters, it is important that the aromatic dicarboxylic acid contain few impurities i.e., be a high purity, typically having a purity of greater than 98%, and desirably greater than about 99.5%. However, in producing the aromatic dicarboxylic acids, a number of impurities or contaminants in the form of the monocarboxylic acid, aldehydes, unoxidized materials, over oxidized materials, catalyst residues, and the like are also produced. Most or all of these contaminants or impurities may affect the usefulness of the aromatic dicarboxylic acid as a monomer for preparing polyesters and polyamides.

For example, in the case of TPA and IPA, carboxybenzaldehyde (CBA) and toluic acid result from the incomplete oxidation of the aromatic dimethyl compound. Both CBA and toluic acid are undesirable since neither the CBA nor the toluic acid have two carboxylic acid groups, both would terminate the chain of a polyester produced from a crude dicarboxylic acid. Advantageously, both CBA and toluic acid are produced in small quantities and are water soluble.

In addition to the CBA and toluic acid impurities, compounds generally known as “fluorenones” are produced. Fluorenones have two carboxylic acid groups, and are therefore not chain terminating. However, fluorenones are yellow. Thus, if fluorenones are present, the polyester produced from the aromatic dicarboxylic acid will appear off color.

In view of the foregoing, it is necessary to purify crude aromatic dicarboxylic acids. Processes for preparing purified aromatic dicarboxylic acid(s) are well known and generally involve at least one crystallization step. For example, U.S. Pat. No. 6,265,608 discloses a process for purifying TPA and IPA by a process that includes the steps of dissolving the products of the oxidizing step in a solvent; hydrogenating the products of the oxidizing step in the presence of a palladium catalyst; and introducing carbon monoxide during the hydrogenation step.

In the case where the oxidation step produces is ophthalic acid, the hydrogenation step hydrogenates the 3-carboxybenzaldehyde to m-toluic acid and 3-hydroxymethyl benzoic acid. In the case were the oxidation step produces terephthalic acid, the hydrogenation step hydrogenates the 4-carboxybenzaldehyde and fluorenones to p-toluic acid and 4-hydroxymethyl benzoic acid.

To obtain purified crystals of TPA and IPA, the crude crystallized product is re-dissolved in a solvent. The solvent is selected so that the impurities have a greater solubility than the crystallized product. As the temperature is reduced, the TPA and IPA recrystallize in a higher purity form, generally having a purity greater than 99 percent.

U.S. Pat. No. 6,054,610 discloses a process for preparing purified TPA and IPA where the crude mixed acids from the oxidation section are re-dissolved in a selective crystallization solvent. The reactor effluent is fed to a series of crystallizers which allow the solids to grow by evaporating the reaction solvent, using stepwise pressure reductions. The crystallized and progressively purified TPA is separated and the filter cake of purified TPA ultimately obtained is washed and soaked with water to remove color and the final trace of the selective crystallization solvent from the TPA product. The filtered crystals are then dried to remove the solvent to a level of less than 0.25% in the resulting crude mixed acid crystals.

U.S. Pat. No. 6,140,534 discloses a method similar to that described in U.S. Pat. No. 6,054,610 for purifying crude IPA from a liquid dispersion containing unreacted starting materials, solvents, products of side reactions and/or other undesired materials. The method includes the steps of filtering the dispersion to form a crude IPA filter cake; dissolving the filter cake in a selective crystallization solvent at an elevated temperature to form a solution; crystallizing purified IPA from the solution in the crystallization solvent; separating the crystallized purified IPA from the solution; and re-dissolving or soaking the washed purified IPA cake at elevated temperature to remove the final traces of the crystallization solvent and obtain the desirable particle sizes and shape. The preferred selective crystallization solvent is N-methyl pyrrolidone. In order to remove the residual solvent trapped in the crystals of the final IPA product, the washed IPA crystals are fed to a high temperature soaker where water is used to partially or completely dissolve the IPA crystals. The IPA crystals are again precipitated or otherwise separated from the soaking water.

In the aforementioned processes, the high purity dicarboxylic acid crystals are typically filtered, dried and transferred to a storage vessel or bin for later shipment or use. A problem not recognized by the prior art is when such purified crystalline materials are stored or placed in large shipping containers, the crystalline aromatic dicarboxylic acid(s) form agglomerates that impede the free flow of material from the storage vessel. Although not to be bound by any theory, it is believed that residual solvent is trapped in the crystals of the purified aromatic dicarboxylic acid product and the drying step, although effective in removing most if not all of the solvent present on the surface of the crystallized product, the drying step does not completely remove the residual solvent trapped in the crystals. In the case were the aromatic dicarboxylic acid is TPA or IPA, water trapped in the crystals is not completely removed. Over a period of time, this water can migrate to the surface of the crystalline structure. The water on the surface of the TPA or IPA causes other crystals of the purified product to agglomerate or form clumps. These aggregates or clumps impede the free flow of the crystallized product out of the storage container and at times may cause bridging or localized plugging problems during unloading. This is especially problematic for unloading bulk containers during times when temperatures are above 75° F. (24° C.) and the relative humidity is above 50%.

Accordingly, there is a need for a method for making aromatic dicarboxylic acid(s) that are high purity, crystalline and free flowing.

### SUMMARY OF THE INVENTION

Briefly, the present invention is a method for making a free-flowing, crystalline, high purity aromatic dicarboxylic acid(s). More specifically, the present invention is an improvement to the method of making crystalline aromatic dicarboxylic acid(s) which utilize a solvent crystallization step and a drying step, the improvement includes the steps of: storing the dried crystallized aromatic dicarboxylic acid for a period of time sufficient for at least a portion of residual solvent retained with the dried crystallized aromatic dicarboxylic acid to reside on the exterior surface of the crystalline aromatic dicarboxylic acid; and subjecting the stored crystallized aromatic dicarboxylic acid to a second drying step by contacting the stored crystallized product with an inert fluid for a time sufficient to remove at least a portion of the residual solvent in the stored crystallized product

It is an object of the present invention to provide a high purity, crystalline aromatic dicarboxylic acid that is substantially free-flowing.

Another object of the present invention to provide a high purity, crystalline aromatic dicarboxylic acid that is substantially free-flowing and easily removed from a storage container.

It is another object of the present invention is to provide a high purity, crystalline aromatic dicarboxylic acid that is substantially free-flowing and easily removed from a storage container when ambient conditions are above 75° F. (24° C.) and the relative humidity is above 50%.

These and other objects and advantages of the present invention will become more apparent to those skilled in the art in view of the following description and the accompanying drawings wherein like parts and objects have similar reference numerals. It is to be understood that the inventive concept is not to be considered limited to the constructions disclosed herein but instead by the scope of the appended claims.

### BRIEF DESCRIPTION OF THE DRAWINGS

FIG. 1 is a schematic view illustrating the prior art wherein loading of a bulk container is from a storage vessel, bin or container.

FIG. 2 is a schematic view illustrating an embodiment of the present invention wherein the stored crystallized aromatic dicarboxylic acid is subjected to a second drying step prior to the loading of the bulk container.

FIG. 3 is a schematic view illustrating another embodiment of the present invention.

### DETAILED DESCRIPTION OF THE INVENTION

The process of the present invention is an improvement to such processes for preparing high purity, crystalline aromatic dicarboxylic acids. The improved process produces high purity, crystalline aromatic dicarboxylic acids that are free-flowing subsequent to purification and particularly crystalline aromatic dicarboxylic acids that are free-flowing from bulk containers. Process for manufacturing crystalline aromatic dicarboxylic acids are well known in the art. Particular examples include processes described in U.S. Pat. Nos. 5,110,984; 5,110,984; 5,481,033; and 4,892,972, to name only a few, for preparing purified crystalline naphthalene dicarboxylic acid, TPA and IPA. The entire disclosures of these patents are incorporated herein by reference. One skilled in the art will understand that the present improvement is applicable to all processes for making crystalline aromatic dicarboxylic acids, such as for example, benzene dicarboxylic acid, naphthalene dicarboxylic acid, bibenzoic acid, terephthalic acid and isophthalic acid that include at least one solvent crystallization step. However, for the sake of brevity and familiarity, the present process is described in terms of a TPA and/or IPA process(es).

Generally, the purified TPA is prepared using at least one solvent crystallization step which may include washing or dissolving the purified TPA with a solvent to remove impurities such as CBA, and toluic acid that are present in the crude product. Suitable solvents include polar solvents selected from N,N-dimethyl acetamide, N,N-dimethyl formamide, N-formyl piperidine, N-alkyl-2-pyrrolidone (such as N-ethyl pyrrolidone), N-mercaptoethyl-2-pyrrolidone, N-methyl-2-thiopyrrolidone, N-hydroxyethyl-2-pyrrolidone, morpholine, N-formyl morpholine, C₁-C₁₂ alcohols, ethers, amines, amides, esters, water and mixtures thereof. Preferably, the solvent is water. When water is the solvent, purification of the aromatic dicarboxylic acid product can be accomplished by dissolving the product in an aqueous solution at an elevated temperature of about 212° F. (100° C.) to about 572° F. (300° C.), then reducing the temperature to about 95° F. (35° C.) to about 248° F. (120° C.), in order to crystallize the aromatic dicarboxylic acid product. The purified product crystals are then separated from the aqueous solution. A solid-liquid separation device, such as a centrifuge or filter, may be employed for separating the crystals from the aqueous solvent. The crystals may be washed with additional solvent within the solid-liquid separation device, or the crystals may be combined with additional solvent and sent to a second solid-liquid separation step. The washed product crystals are then passed to a dryer and heated to a temperature of about 212° F. (100° C.) to about 356° F. (180° C.) to remove liquid remaining on the crystals.

A substantial portion of the TPA or IPA crystalline product has an average crystal size of from about 50 to 400 microns, preferably from 50 to 300 microns, more preferably from 50 to about 200 microns and most preferably from 100 to 200 microns. The crystal size of the product is determined in accordance with the procedure described below.

A four to eight ounce (113-226 gram) container is partially filled with a dry sample of the test material. The sample is homogenized in the container. The instrument used is a Malvern laser diffraction dry powder particle size analyzer available from Malvern Insturments Ltd., United Kingdom. The sample is fed at a rate that delivers an adequate amount of sample material but does not saturate the detector. Statistical results of particle size and distribution are calculated.

As used herein, the term “substantial portion” means that greater than 50 percent, preferably greater than 60 percent, more preferably greater than 80 percent and most preferably greater than 90 percent of the crystalline product has an average crystal size described above. Desirably, the purity of the crystalline product is greater than about 98%, preferably greater than 99% and more preferably greater than 99.5% of the desired dicarboxylic acid.

Referring to FIG. 1, the prior art is illustrated. The crystalline dicarboxylic acid is prepared by a known process, dried and transferred via line 10 to a storage vessel 20. The crystallized product is then transferred from the storage bin 20 via line 25 to the bulk container, illustrated in the drawing as a rail car 30.

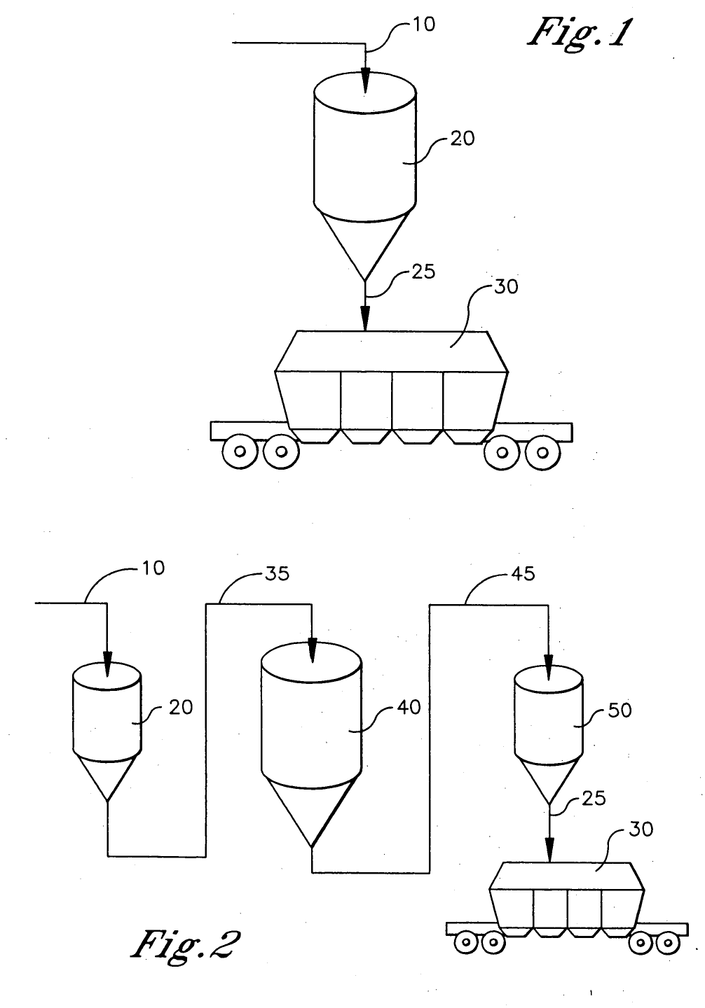

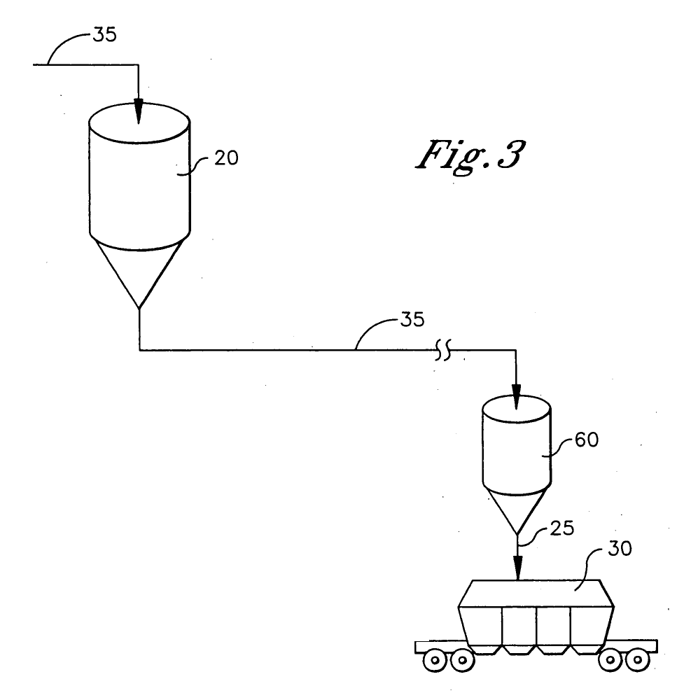

An improvement has surprisingly been discovered in preparing a high purity, crystalline dicarboxylic acid product that is free-flowing when removed from such bulk containers. Referring to FIG. 2, the crystalline dicarboxylic acid product is prepared by a known process, dried and transferred via line 10 to a storage vessel 20. In accordance with the present invention, the dried, crystallized product is stored in vessel 20 of a suitable volume for a period of time sufficient for at least a portion of residual solvent carried with the crystallized product to reside on the exterior surface of the crystalline product. Desirably, the crystalline product is stored for a period of time of from about 1 hour to about 5 days, preferably from about 1 hour to about 3 days, more preferably from about 1 hour to about 24 hours and most preferably from about 1 hour to about 8 hours. Although it is possible to contact the stored crystalline product with an inert gas to remove any residual solvent, it has been observed that when the product has an average crystal size of greater than about 50 microns, channels are formed allowing the inert gas to pass through the stored crystalline product without significant removal of the residual solvent. Accordingly, it is desirable for the stored crystalline product to not be disturbed during this period of time.

After a sufficient period of time for at least a portion of residual solvent to migrate from the crystalline structure and reside on the exterior surface of the crystalline product, the crystalline product is transferred from storage vessel 20 via line 35 to a dryer 40. The crystalline product is then contacted with an inert fluid adapted to remove the residual solvent for a period of time sufficient to remove at least a portion and desirably all of the residual solvent residing on the surface of the crystallized product. Desirably, the inert fluid is a nitrogen containing gas. When the residual solvent is water, it is desired that the relative humidity of the gas be less than about 50%. Preferably, the relative humidity of the gas is less than about 30%, more preferably the relative humidity of the gas is less than about 10%, and most preferably, the gas is bone dry. As used herein, the term “bone dry” means having a relative humidity of less than about 3%. The inert fluid contacts the crystallized product for a period of time sufficient to remove at least a portion of residual solvent. One skilled in the art will recognize that the amount of time necessary to effect removal of the residual solvent will vary depending upon several factors, such as: the size of the dryer; the dryness of the inert fluid; and the amount of residual solvent on the crystalline product. However, typical times necessary range from about 5 seconds to about 30 minutes, preferably from about 10 seconds to about 10 minutes, and more preferably from about 30 seconds to about 5 minutes.

The dryer 40 can be any type of dryer known to those skilled in the art, such as rotary kiln dryers, fluidized bed dryers, steam tube dryers vacuum dryers, and the like. The crystallized product is then transferred from the dryer 40 via line 45 to a collection vessel 50, such as a bag-house or second storage bin for loading of the bulk container 30.

Referring to FIG. 3, a second embodiment of the present invention is illustrated. The crystallized dicarboxylic acid is prepared in accordance with a known process, dried and is transferred via line 10 to a storage vessel 20. The dried, crystallized product is stored in the vessel 20 for a period of time sufficient for at least a portion of the residual solvent carried with the crystallized product to reside on the exterior surface of the crystalline product. Afterwards, the crystalline product is pneumatically transferred from storage vessel 20 via line 35 to a collection vessel 60, such as a bag-house or second storage bin for loading of the bulk container 30. In accordance with this embodiment of the invention, the transfer line appropriately sized in diameter and length so that inert fluid utilized to transport the crystalline product contacts the crystallized product for a period of time, as described above, sufficient to remove at least a portion and desirably all of residual solvent. To effectively transport and contact crystalline product, the volume to volume ratio of inert fluid to crystallized product used in the pneumatic transfer is from about 1:1 to about 100:1, preferably from about 2:1 to 10:1 and more preferably from about 4:1.

The present invention is illustrated in greater detail by the specific examples presented below. It is to be understood that these examples are illustrative embodiments and are not intended to be limiting of the invention, but rather are to be construed broadly within the scope and content of the appended claims.

### COMPARATIVE EXAMPLE 1

During a period of time, a total of 38 railroad cars were loaded with an average of about 188,945 lbs (85,705 kg) of purified isophthalic acid. The isophthalic acid was not prepared in accordance with the process of the present invention. The gases released during loading were measured to have a relative humidity of greater than 70%. The product in each rail cars had an average storage time, as measured from the time loaded to the time unloaded, of about 1025 hours. An average of 14,352 lbs (6,510 kg) of product in each railcar was returned due to unloading difficulties.

### EXAMPLE 2

During a period of time, a total of 77 railroad cars were loaded with an average of about 187,534 lbs (85,065 kg) of purified isophthalic acid. The isophthalic acid was prepared in accordance with the process of the present invention. The gases released during loading were measured to have a relative humidity of less than 10%. The product in each rail cars had an average storage time, as measured from the time loaded to the time unloaded, of about 1500 hours. An average of 1870 lbs (848 kg) of product in each railcar was returned due to unloading difficulties.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

1. In a process for making a high purity, crystalline aromatic dicarboxylic acid product which includes crystallizing the product and wherein the crystallized product is thereafter subjected to a drying step, wherein the improvement comprises:
a. storing said crystallized product for a period of time sufficient for at least a portion of residual solvent carried with said dried crystallized product to reside on a surface of the crystallized product; and
b. contacting the stored crystallized product with an inert fluid for a time sufficient to remove at least a portion of the residual solvent residing on the surface of the crystalline product.

2. The process of claim 1 wherein said crystalline aromatic dicarboxylic acid is selected from the group consisting of benzene dicarboxylic acid, naphthalene dicarboxylic acid, bibenzoic acid, terephthalic acid, and isophthalic acid.

3. The process of claim 2 wherein said crystalline aromatic dicarboxylic acid is terephthalic acid.

4. The process of claim 2 wherein said crystalline aromatic dicarboxylic acid is isophthalic acid.

5. The process of claim 1 wherein said solvent is selected from the group consisting of N,N-dimethyl acetamide, N,N-dimethyl formamide, N-formyl piperidine, N-alkyl-2-pyrrolidone (such as N-ethyl pyrrolidone), N-mercaptoethyl-2-pyrrolidone, N-methyl-2-thiopyrrolidone, N-hydroxyethyl-2-pyrrolidone, morpholine, N-formyl morpholine, C₁-C₁₂ alcohols, ethers, amines, amides, esters, water and mixtures thereof.

6. The process of claim 1 wherein said solvent is water.

7. The process of claim 1 wherein a substantial portion of said crystalline product has an average crystal size of from 50 to about 400 microns.

8. The process of claim 1 wherein a substantial portion of said crystalline product has an average crystal size of from 50 to about 300 microns.

9. The process of claim 1 wherein a substantial portion of said crystalline product has an average crystal size of from 50 to about 200 microns.

10. The process of claim 1 wherein a substantial portion of said crystalline product has an average crystal size of from about 100 to about 200 microns.

11. The process of any one of claims 7 to 10 wherein said substantial portion is greater than 50 percent.

12. The process of claim 11 wherein said substantial portion is greater than 60 percent.

13. The process of claim 11 wherein said substantial portion is greater than 80 percent.

14. The process of claim 11 wherein said substantial portion is greater than 90 percent.

15. The process of claim 1 wherein said crystalline aromatic dicarboxylic acid has a purity greater than about 98%.

16. The process of claim 1 wherein said crystalline aromatic dicarboxylic acid has a purity greater than about 99%.

17. The process of claim 1 wherein said crystalline aromatic dicarboxylic acid has a purity greater than about 99.5%.

18. The process of claim 1 wherein said inert fluid comprises a nitrogen containing gas.

19. The process of claim 1 wherein contacting said stored crystallized product with said inert fluid comprises pneumatic conveying of the stored crystallized product.

20. The process of claim 1 wherein said inert fluid contacts said crystallized aromatic dicarboxylic acid for a period of from about 5 seconds to about 30 minutes.

21. The process of claim 1 wherein said inert fluid contacts said crystallized aromatic dicarboxylic acid for a period of from about 10 seconds to about 10 minutes.

22. The process of claim 1 wherein said inert fluid contacts said crystallized aromatic dicarboxylic acid for a period of from about 30 seconds to about 5 minutes.

23. The process of claim 1 wherein contacting said stored crystallized product with said inert fluid with comprises drying the stored crystallized product in a staged dryer.

24. In a process for making a high purity, crystalline aromatic dicarboxylic acid product selected from the group consisting of naphthalene dicarboxylic acid, terephthalic acid, and isophthalic acid, the process which includes crystallizing the product and wherein the crystallized product is thereafter subjected to a drying step, wherein the improvement comprises:
a. storing said crystallized product for a period of time sufficient for at least a portion of residual solvent carried with said dried crystallized product to reside on a surface of the crystallized product; and
b. contacting the stored crystallized product with an inert fluid for a time of from 5 seconds to about 30 minutes.

25. The process of claim 24 wherein said crystalline aromatic dicarboxylic acid is terephthalic acid.

26. The process of claim 24 wherein said crystalline aromatic dicarboxylic acid is. isophthalic acid.

27. The process of claim 24 wherein said solvent is water.

28. The process of claim 24 wherein greater than 50% said crystalline aromatic dicarboxylic acid has an average crystal size of from about 100 to about 200 microns.

29. The process of claim 24 wherein greater than 60% said crystalline aromatic dicarboxylic acid has an average crystal size of from about 100 to about 200 microns.

30. The process of claim 24 wherein greater than 80% said crystalline aromatic dicarboxylic acid has an average crystal size of from about 100 to about 200 microns.

31. The process of claim 24 wherein greater than 90% said crystalline aromatic dicarboxylic acid has an average crystal size of from about 100 to about 200 microns.

32. The process of claim 24 wherein said crystalline aromatic dicarboxylic acid has a purity greater than about 99.5%.

33. The process of claim 24 wherein contacting said stored crystallized product with said inert fluid comprises pneumatic conveying of the stored crystallized product.

34. In a process for making a high purity, crystalline aromatic dicarboxylic acid product selected from the group consisting of terephthalic acid, and isophthalic acid, the process which includes crystallizing the product from an aqueous solvent and wherein the crystallized product is thereafter subjected to a drying step, wherein the improvement comprises:
a. storing said crystallized product for a period of time sufficient for at least a portion of residual solvent carried with said dried crystallized product to reside on a surface of the crystalline product; and
b. contacting the stored crystallized product with an inert fluid for a time of from 5 seconds to about 30 minutes.

35. The process of claim 34 wherein greater than 50% of said crystalline product has an average crystal size of from 50 to 400 microns.

36. The process of claim 34 wherein greater than 80% of said crystalline product has an average crystal size of from 100 to 200 microns.

37. The process of claim 34 wherein said inert gas contacts said. crystallized aromatic dicarboxylic acid for a period of from about 10 seconds to about 10 minutes.

38. The process of claim 34 wherein said inert gas contacts said crystallized aromatic dicarboxylic acid for a period of from about 30 seconds to about 5 minutes.

39. The process of claim 34 wherein contacting said stored crystallized product with said inert fluid comprises pneumatic conveying of the stored crystallized product.

40. The process of claim 34 wherein said inert fluid includes a nitrogen containing gas.
